(19) United States
(12) Patent Application Publication
(10) Pub. No.: US 2011/0071628 A1
(43) Pub. Date: Mar. 24, 2011

(54) ACCOMMODATIVE INTRAOCULAR LENS

(75) Inventors: Yossi GROSS, Moshav Mazor (IL); Nir BETSER, Yehud (IL)

(73) Assignee: RAINBOW MEDICAL LTD., Herzeliya (IL)

(21) Appl. No.: 12/566,029

(22) Filed: Sep. 24, 2009

(51) Int. Cl. A61F 2/16

(52) U.S. Cl. 623/6.51

(57) ABSTRACT

An accommodating intraocular lens (AIOL) implant includes at least an anterior floating lens complex and a posterior lens complex, each of which comprises one or more optical elements, and a frame comprising one or more levers, which are coupled to the frame and the anterior floating lens complex. The levers are configured to leverage motion of the frame to move the anterior floating lens complex with respect to the posterior lens complex. Other embodiments are also described.

### FIELD OF THE INVENTION

The present invention relates generally to implantable medical devices, and specifically to intraocular lenses.

### BACKGROUND OF THE INVENTION

Accommodating intraocular lenses (AIOLs) allow the eye to focus at different distances. The Crystalens® (Bausch & Lomb, Rochester, N.Y., USA) is an AIOL that has received FDA approval in the United States.

The following references may be of interest:

U.S. Pat. No. 7,416,562 to Gross McLeod SD et al., “Synchrony dual-optic accommodating intraocular lens Part 1: Optical and biomechanical principles and design considerations,” J Cataract Refract Surg. 2007; 33:37-46 Ossma IL et al., “Synchrony Dual-Optic Accommodating Intraocular Lens Part 2: Pilot Clinical Evaluation,” J Cataract Refract Surg. 2007; 33:47-52.

### SUMMARY OF THE INVENTION

In embodiments of the present invention, an accommodative intraocular lens (AIOL) implant comprises posterior and anterior lens complexes coupled to a frame. The AIOL implant is configured such that the width (in the anterior-posterior direction) of the frame changes in response to the natural accommodation mechanism of the eye. This change in width changes the distance between the anterior lens complex and the posterior lens complex, thereby adjusting the focal length of the AIOL implant. The frame comprises one or more levers, which magnify the relatively small change in the width of the frame caused by the natural change in the shape of the capsular bag, in order to move the anterior lens complex a greater distance with respect to the posterior lens complex. Because of this distance magnification, the AIOL implant provides a high level of accommodation that mimics that of the natural eye.

The AIOL implant's accommodation typically provides a continuous range of focus, including near, distance, and intermediate distances. The AIOL implant exploits the natural accommodation mechanism of the eye, which reacts in order to sharpen the image on the retina. The AIOL implant thus typically reduces the need for glasses, which are generally required by patients with conventional IOLs. The AIOL implant is typically implanted in the eye after natural lens removal because of cataract, or for Refractive Lens Exchange (RLE), using well-known IOL implantation techniques, including making a small incision.

For some applications, the frame of the AIOL implant is configured to stretch the natural capsular bag of the eye toward its natural, fully functional shape. Assuming this reduced-diameter natural shape increases the tension of the zonular fibers, and thus allows the zonular fibers and ciliary muscle to function normally, and interact with the AIOL implant as they would with the natural lens. As a result, the AIOL implant to a large extent restores the eye's natural accommodation mechanism. For some applications, the frame comprises a plurality of wings, which extend radially outward from the interior of the AIOL implant, and are configured to help restore the natural, fully functional shape of the capsular bag.

There is therefore provided, in accordance with an embodiment of the present invention, apparatus including an accommodating intraocular lens (AIOL) implant, which includes:

at least an anterior floating lens complex and a posterior lens complex, each of which includes one or more optical elements; and

a frame including one or more levers, which are coupled to the frame and the anterior floating lens complex, and are configured to leverage motion of the frame to move the anterior floating lens complex with respect to the posterior lens complex.

Typically, the levers are coupled to the frame and the anterior floating lens complex such that the levers move the anterior floating lens complex by a first distance with respect to the posterior lens complex, when an anterior-posterior width of the frame increases by a second distance, the first distance greater than the second distance.

For some applications, the AIOL implant is configured to decrease a diameter of a natural capsular bag of an eye in which the AIOL implant is implanted. For example, the frame may include a plurality of wings, which are configured to decrease the diameter of the natural capsular bag.

For some applications, the frame includes an accommodating lens holder, arranged such that the levers are coupled to the anterior floating lens complex indirectly via the accommodating lens holder. Alternatively, the levers are coupled directly to the anterior floating lens complex.

For some applications, the frame includes an anterior support structure; a posterior support structure, to which the posterior lens complex is coupled; and one or more links, which couple the anterior support structure to the posterior support structure, and provide a variable distance between the support structures. For some applications, the levers are coupled to respective ones of the links. Typically, the levers are coupled to the frame and the anterior floating lens complex such that the levers move the anterior floating lens complex by a first distance with respect to the posterior support structure, when the anterior support structure moves a second distance with respect to the posterior support structure, the first distance greater than the second distance. For some applications, the links are configured to provide spring functionality that tends to push the anterior support structure away from the posterior support structure.

For some applications, the frame is configured to assume locked and unlocked states. For some applications, the frame is configured to automatically transition from the locked state to the unlocked state after a period of time after implantation of the AIOL implant in an eye.

The present invention will be more fully understood from the following detailed description of embodiments thereof, taken together with the drawings, in which:

### BRIEF DESCRIPTION OF THE DRAWINGS

FIGS. 1A-B are schematic isometric views of an accommodative intraocular lens (AIOL) implant, in non-accommodated and fully-accommodated states, respectively, in accordance with an embodiment of the present invention;

FIGS. 2A-B are schematic side views showing the AIOL implant of FIGS. 1A-B implanted in a natural capsular bag of the eye, in non-accommodated and fully-accommodated states, respectively;

FIGS. 3A-B are simplified side views showing only a portion of the AIOL implant of FIGS. 1A-B, for clarity of illustration;

FIGS. 4A-B are schematic side views of another configuration of the AIOL implant of FIGS. 1A-B implanted in the natural capsular bag, in non-accommodated and fully-accommodated states, respectively, in accordance with an application of the present invention;

FIGS. 5A-B are schematic side views of yet another configuration of the AIOL implant of FIGS. 1A-B implanted in the natural capsular bag, in non-accommodated and fully-accommodated states, respectively, in accordance with an application of the present invention;

FIGS. 6A-B are schematic isometric views of still another configuration of the AIOL implant of FIGS. 1A-B, in non-accommodated and fully-accommodated states, respectively, in accordance with an application of the present invention;

FIGS. 7A-B are schematic side views of another configuration of the AIOL implant of FIGS. 1A-B, in non-accommodated and fully-accommodated states, respectively, in accordance with an application of the present invention; and

FIGS. 8A-B are schematic side views of yet another configuration of the AIOL implant of FIGS. 1A-B implanted in the natural capsular bag, in non-accommodated and fully-accommodated states, respectively, in accordance with an application of the present invention.

### DETAILED DESCRIPTION OF EMBODIMENTS

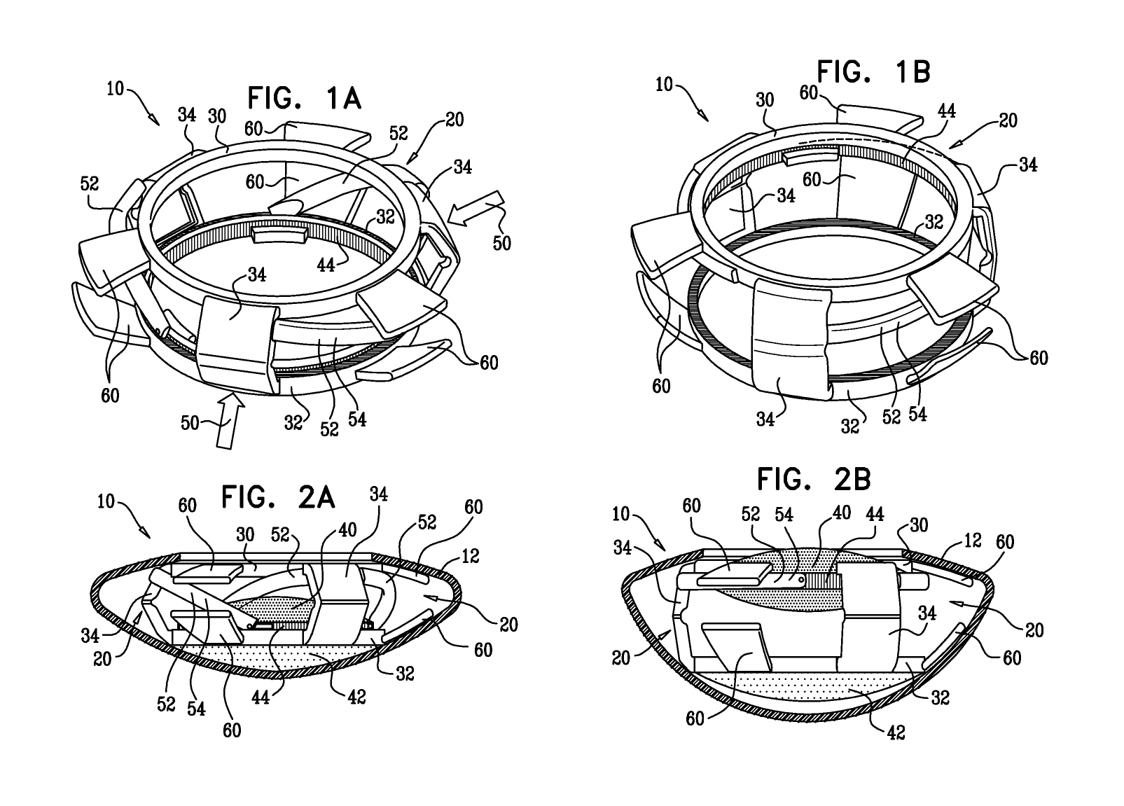

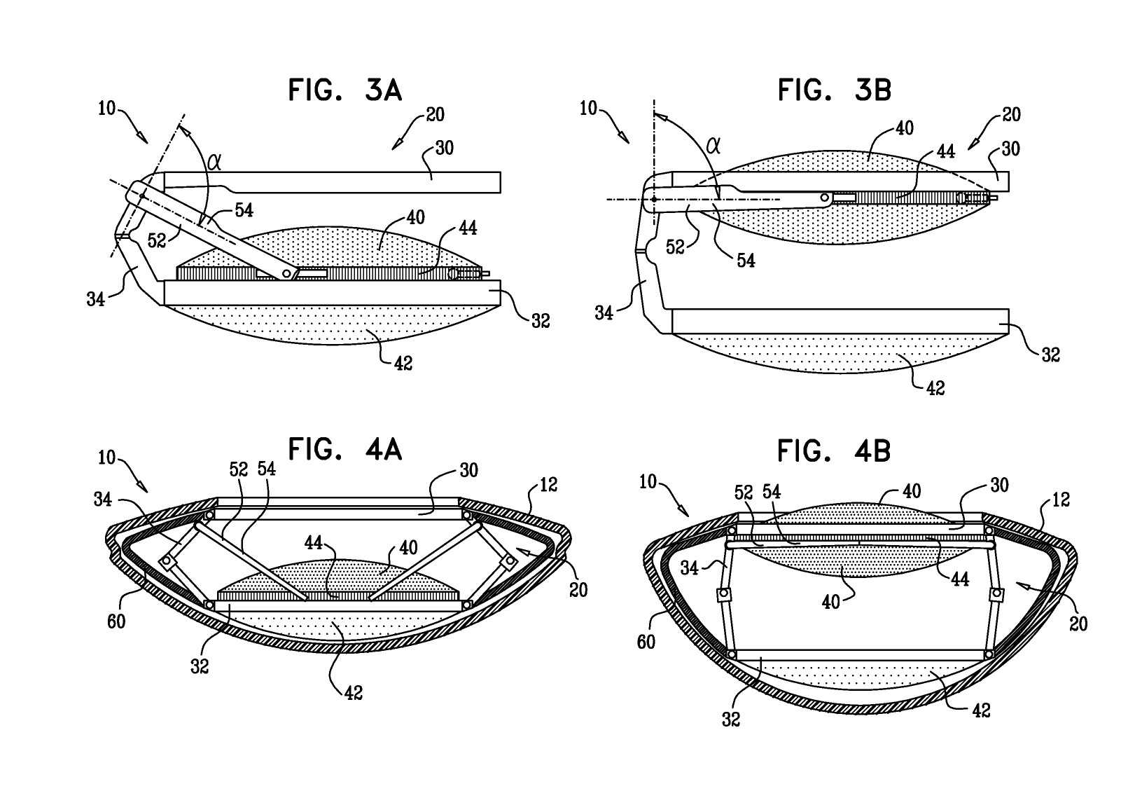

FIGS. 1A-B, 2A-B, and 3A-B are schematic illustrations of an accommodative intraocular lens (AIOL) implant 10, in accordance with an embodiment of the present invention. FIGS. 1A-B are isometric views of the AIOL implant. FIGS. 2A-B are side views showing the AIOL implant implanted in a natural capsular bag 12 of the eye. FIGS. 3A-B are simplified side views showing only a portion of the AIOL implant for clarity of illustration.

FIGS. 1A, 2A, and 3A show AIOL implant 10 in a non-accommodated state, while FIGS. 1B, 2B, and 3B show the AIOL implant in a fully-accommodated state. Although only these two states are shown in these and the other figures, AIOL implant 10 is typically configured to assume a continuous range of accommodation between the non-accommodated state and the fully-accommodated state. The fully-accommodated state provides near vision, the non-accommodated state provides distance vision, and partially-accommodated states therebetween provide intermediate vision. The AIOL implant is configured to reach the fully-accommodated state responsively to the natural accommodation mechanism of the eye, without the need for external power.

AIOL implant 10 comprises a frame 20, which typically comprises anterior and posterior support structures 30 and 32. For some applications, both of the support structures are shaped as rings, which are typically concentric. Support structures 30 and 32 are coupled together by one or more links 34 (e.g., one to three, or four or more), which are coupled to the support structures typically such that the links articulate with the support structures. The links are configured to provide a variable distance between the two support structures, as described hereinbelow. To this end, the links are flexible, include flexible portions, and/or include hinges.

AIOL implant 10 further comprises at least an anterior floating lens complex 40 and a posterior lens complex 42. (The lens complexes are shown in FIGS. 2A-B and 3A-B, but are omitted in FIGS. 1A-B for clarity of illustration.) Posterior lens complex 42 is coupled to posterior support structure 32, and remains generally fixed with respect to the posterior of the eye during accommodation of the AIOL implant. For some applications, as shown in FIGS. 1A-B, 2A-B, and 3A-B, anterior floating lens complex 40 is coupled to an accommodating lens holder 44, which may be shaped as a ring.

The AIOL implant is configured such that anterior floating lens complex 40 (and accommodating lens holder 44, if provided) moves with respect to posterior lens complex 42 (and posterior support structure 32) in response to the natural accommodation mechanism of the eye. The natural accommodation mechanism of the eye changes the shape of capsular bag 12, as shown in FIGS. 2A-B. In the non-accommodated state shown in FIG. 2A, the ciliary muscle is relaxed and the zonular fibers are thus tensed, causing the capsular bag to assume a relatively narrow width (in the anterior-posterior direction) and relatively large diameter. As used herein, including in the claims, the diameter of the capsular bag means the greatest diameter of the capsular bag when viewed from its posterior aspect. This shape of the capsular bag squeezes the AIOL implant in the anterior-posterior direction. In contrast, in the fully-accommodated state shown in FIG. 2B, the ciliary muscle contracts, thereby releasing the tension of the zonular fibers on the capsular bag, causing the capsular bag to assume a relatively large width and relative small diameter. This shape of the capsular bag allows the AIOL implant to expand in the anterior-posterior direction.

As the width of the capsular bag changes, as described above, anterior support structure 30 moves with respect to posterior support structure 32, thereby changing the distance between the support structures. As described above, frame 20 typically comprises one or more links 34, which couple anterior support structure 30 to posterior support structure 32. Links 34 flex and assume a straighter, longer shape, as the support structures move away from each other. For some applications, links 34 are configured to function as springs that tend to push anterior support structure 30 away from posterior support structure 32 when the natural capsular bag is not applying a force that squeezes the support structures together. Alternatively or additionally, frame 20 comprises one or more separate springs that are configured to widen the AIOL implant (configuration not shown).

When the width of AIOL implant 10 changes, one or more elements of frame 20 move anterior floating lens complex 40 (and accommodating lens holder 44, if provided) with respect to posterior lens complex 42 (and posterior support structure 32). The elements of frame 20 that cause this relative motion typically include one or more levers 52 (e.g., between one and three, or four or more). Typically, one end of each of the levers is attached to one of links 34, and the other end is attached to anterior floating lens complex 40 (either directly or via accommodating lens holder 44, if provided). The levers magnify the relatively small change in the width of the frame and in the distance between support structures 30 and 32, in order to move accommodating lens holder 44 by a greater distance with respect to posterior support structure 32. Because of this distance magnification, the AIOL implant provides a high level of accommodation that mimics that of the natural eye. The levers are typically attached to the links at respective points along the links that result in optimal angular change due to the straightening of the links, and a resulting optimal shifting of the anterior floating lens complex.

Typically, anterior floating lens complex 40 (and accommodating lens holder 44, if provided) moves a first distance with respect to posterior support structure 32, when anterior support structure 30 moves a second distance with respect to posterior support structure 32, which first distance is greater than the second distance.

For some applications, as shown in FIGS. 1A-B, 2A-B, and 3A-B, each of levers 52 is defined by an arm 54 and an anterior portion of one of links 34. The arm and the anterior portion of the link are coupled together at a fixed angle α. For some applications, the levers are coupled to accommodating lens holder 44, such that the levers are indirectly coupled to anterior floating lens complex 40. For other applications, the levers are coupled to anterior floating lens complex 40 directly or via a small joining mechanism. In these latter applications, frame 20 does not necessarily comprise accommodating lens holder 44. Typically, the levers are coupled to lens holder 44 or lens complex 40 such that the levers articulate with the holder or lens complex. The points of connection of the levers with the holder or lens complex are optimized to provide an adequate shift of the floating lens complex in response to the natural accommodation of the eye.

The anterior and posterior movement of anterior floating lens complex 40 changes the distance between the lens complexes, thereby adjusting the focal length of the AIOL implant. In the fully-accommodated state, which provides near vision, frame 20 is relatively wide (in the anterior-posterior direction), with a large separation between the anterior and posterior lens complexes, creating a large free space between the complexes. In the non-accommodated state, which provides distance vision, the frame is relatively narrow, with a small separation between anterior and posterior complexes. Anterior floating lens complex 40 typically shifts at least 1 mm between the non-accommodated and fully-accommodated states.

Anterior floating lens complex 40 moves within an interior space of frame 20, which is typically open to the natural fluid within the eye. The floating lens complex is configured to create minimum drag during movement, while maintaining the optical performance of the combined lens structure. For example, the floating lens complex may have a smooth shape, and/or may be coated with a hydrophobic coating such as silicone.

Typically, the lens complexes are configured to together create an optical structure having a total power that varies between +15 D and +25 D, as selected by the physician implanting the AIOL implant. For some applications, the shift of anterior floating lens complex changes the optical power of the combined optical structure in accordance with the following equation:

ΔDc≈(Dm/13)Δs (1)

in which ΔDc is the change in conjugation power of the eye, Dm is the dioptric power of the moving lens complex, and As is the change in lens complex position expressed in millimeters. Thus, the greater the optical power of anterior floating lens complex 40, the greater the change in optical power of the combined optical structure.

Each of lens complexes 40 and 42 comprises one or more optical elements, such as lenses, fixed power optics, convex lenses, concave lenses, biconvex lenses, biconcave lenses, spherical lenses, aspheric lenses, astigmatic lenses, deformable optics, aberration free optics, doublets, triplets, filtered optics, or combinations of these lenses, as is known in the optical arts. Typically, each of lens complexes 40 and 42 comprises at least one lens. For some applications, each of the lens complexes comprises exactly one lens element. For some applications, one or more of lens complexes 40 are attached to frame 20 during manufacture. Alternatively or additionally, one or more of the lens complexes may be attached by a healthcare worker either prior to or during the implantation procedure, such as to provide the lens complex most appropriate for the particular patient.

For some applications, frame 20 further comprises a plurality of wings 60, which extend radially outward from the interior of AIOL implant 10. (The wings are shown in FIGS. 1A-B and 2A-B, but omitted in FIGS. 3A-B for clarity of illustration.) Wings 60 are configured to help stretch capsular bag 12 toward its natural, fully functional shape. Assuming this reduced-diameter natural shape increases the tension of the zonular fibers, and thus allows the zonular fibers and ciliary muscle to function normally, and interact with AIOL implant 10 as they would with the natural lens. Alternatively or additionally, the AIOL implant increases the width (in the anterior-posterior direction) of the capsular bag, which reduces the diameter of the capsular bag, and increases the tension of the zonular fibers. As a result, AIOL implant 10 to a large extent restores the eye's natural accommodation mechanism. Wings 60 function as haptics, which hold AIOL implant 10 in place within the capsular bag.

For some applications, a first portion of wings 60 are coupled to anterior support structure 30, and a second portion of the wings are coupled to posterior support structure 32. The wings coupled to the anterior support structure typically extend radially outward in a posterior direction, while the wings coupled to the posterior support structure typically extend radially outward in an anterior direction. For some applications, the wings are provided in pairs, each of which includes one wing coupled to anterior support structure 30, and one wing coupled to posterior support structure 32, generally aligned with each other, as shown in FIG. 1A-B, 2A-B, and 3A-B. Alternatively, the wings are offset from one another around frame 20. For some applications, the wings are generally shaped as tabs.

For some applications, frame 20 is configured to have locked and non-locked states. For example, in the locked state, the frame may be held in the non-accommodated state, or close thereto. Alternatively, the frame is held in a different position in the locked state. AIOL implant 10 is implanted in the locked state, in order to control the capability of accommodation and/or to optimize the span of accommodation and/or tense the zonular fibers. After implantation, the lock is disengaged, allowing the AIOL implant to accommodate by exploiting the eye's natural accommodation mechanism. For some applications, the locking mechanism is configured to automatically disengage a short time after implantation, e.g., between a few minutes and a few weeks, or longer, after implantation, in order to allow time for the eye to become accommodated to the implant. For some applications, the locking mechanism is implemented using absorbable sutures and/or biodegradable medical adhesives, or by the structure of frame 20. Alternatively, for example, laser energy can be applied to the implant to release the lock.

For some applications, AIOL implant 10 is fully pre-assembled or modularly assembled prior to, during, or after implantation. AIOL implant 10 typically comprises biocompatible materials, such as silicone, acrylic, Poly(methyl methacrylate) (PMMA), Nitinol, or platinum.

Upon implantation of some IOLs, the eye's natural accommodation may be lost over time, with a resultant loss in tension of the zonules, thereby affecting the ability of the zonules to focus the lens. For some applications, AIOL implant 10 comprises mechanical means for reducing or preventing this slackening of the zonules. For some applications, AIOL implant 10 implements one or more of the techniques described in U.S. Pat. No. 7,416,562 to Gross, which is incorporated herein by reference.

FIGS. 4A-B are schematic side views of another configuration of AIOL implant 10 implanted in natural capsular bag 12, in non-accommodated and fully-accommodated states, respectively, in accordance with an application of the present invention. For some applications, as shown in FIGS. 4A-B, links 34 are hinged. Alternatively or additionally, for some applications, a single bent wing 60 serves in place of a pair of wings 60 described hereinabove with reference to FIGS. 1A-B, 2A-B, and 3A-B.

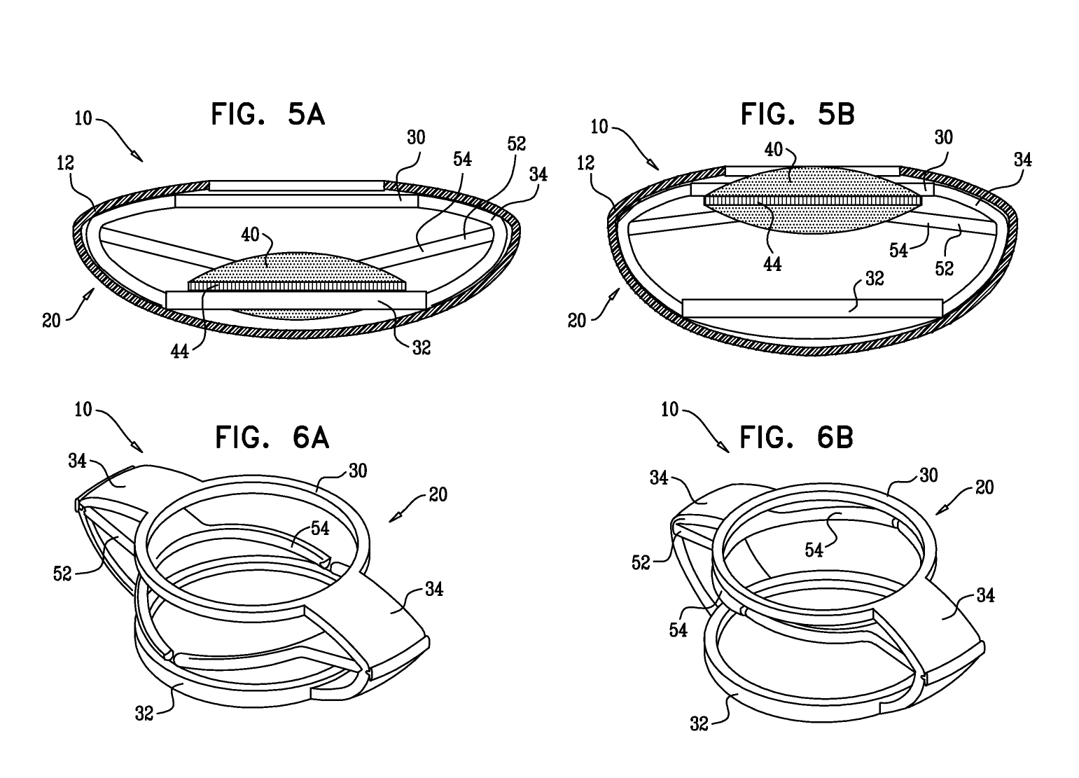

FIGS. 5A-B are schematic side views of yet another configuration of AIOL implant 10 implanted in natural capsular bag 12, in non-accommodated and fully-accommodated states, respectively, in accordance with an application of the present invention.

FIGS. 6A-B are schematic isometric views of still another configuration of AIOL implant 10, in non-accommodated and fully-accommodated states, respectively, in accordance with an application of the present invention. For clarity of illustration, lens complexes 40 and 42 are not shown. In this configuration, levers 52 are directly coupled to anterior floating lens complex 40. Each lever is shaped so as to define two arms 54, which are curved to generally match the shape of the circumference of anterior floating lens complex 40, so as to hold the floating lens complex.

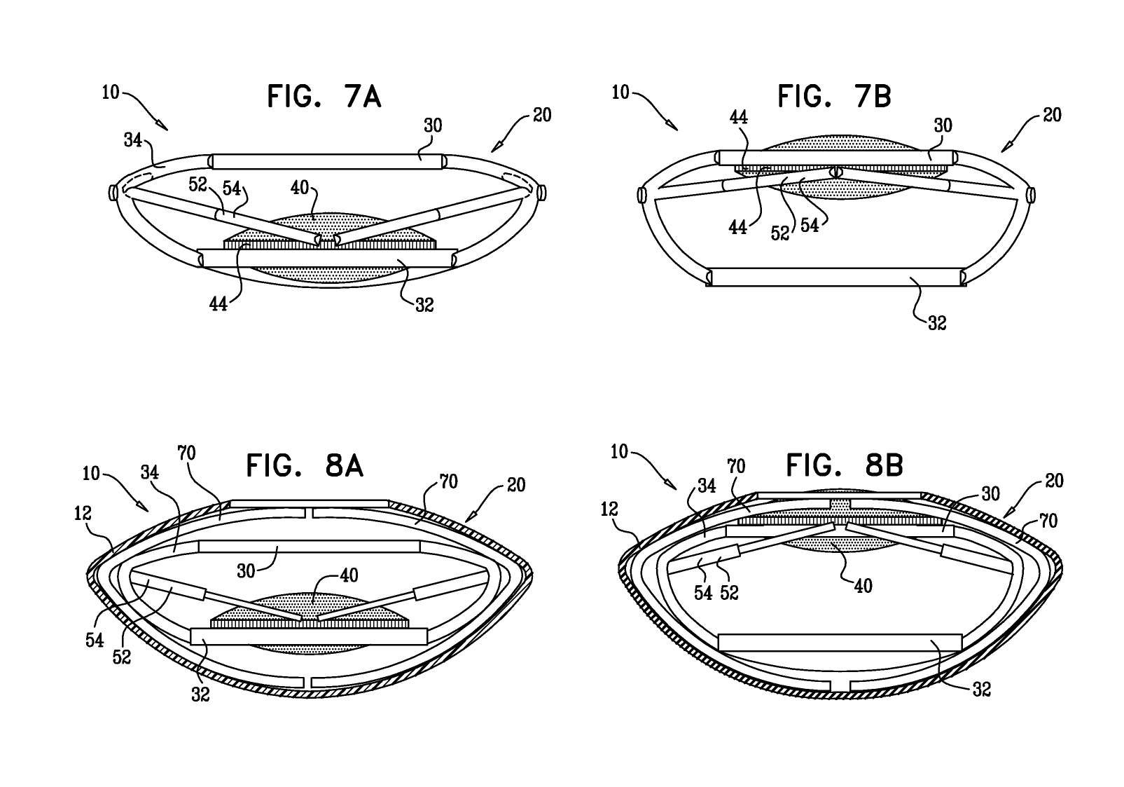

FIGS. 7A-B are schematic side views of another configuration of AIOL implant 10, in non-accommodated and fully-accommodated states, respectively, in accordance with an application of the present invention.

FIGS. 8A-B are schematic side views of yet another configuration of AIOL implant 10 implanted in natural capsular bag 12, in non-accommodated and fully-accommodated states, respectively, in accordance with an application of the present invention. The figures show the floating lens complex shift caused by the reshaping of capsular bag 12 during accommodation.

In this configuration, frame 20 comprises one or more (e.g., two) wings 70, which are configured to help stretch capsular bag 12 toward its natural, fully functional shape. Assuming this reduced-diameter natural shape increases the tension of the zonular fibers, and thus allows the zonular fibers and ciliary muscle to function normally, and interact with AIOL implant 10 as they would with the natural lens. For some applications, each of wings 70 extends in both anterior and posterior directions. Alternatively, the wings extend in only the anterior direction, or only the posterior direction (configuration not shown). For some applications, the wings comprise curved rods (as shown). For applications in which the wings at least in part cover the anterior aspect of capsular bag 12, the wings are typically forked so as to not interfere with vision (e.g., so as not to substantially block the opening in the anterior aspect of the capsular bag).

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising an accommodating intraocular lens (AIOL) implant, which comprises:
at least an anterior floating lens complex and a posterior lens complex, each of which comprises one or more optical elements; and
a frame comprising one or more levers, which are coupled to the frame and the anterior floating lens complex, and are configured to leverage motion of the frame to move the anterior floating lens complex with respect to the posterior lens complex.

2. The apparatus according to claim 1, wherein the levers are coupled to the frame and the anterior floating lens complex such that the levers move the anterior floating lens complex by a first distance with respect to the posterior lens complex, when an anterior-posterior width of the frame increases by a second distance, the first distance greater than the second distance.

3. The apparatus according to claim 1, wherein the AIOL implant is configured to decrease a diameter of a natural capsular bag of an eye in which the AIOL implant is implanted.

4. The apparatus according to claim 3, wherein the frame comprises a plurality of wings, which are configured to decrease the diameter of the natural capsular bag.

5. The apparatus according to claim 1, wherein the frame comprises an accommodating lens holder, arranged such that the levers are coupled to the anterior floating lens complex indirectly via the accommodating lens holder.

6. The apparatus according to claim 1, wherein the levers are coupled directly to the anterior floating lens complex.

7. The apparatus according to claim 1, wherein the frame comprises:
an anterior support structure;
a posterior support structure, to which the posterior lens complex is coupled; and
one or more links, which couple the anterior support structure to the posterior support structure, and provide a variable distance between the support structures.

8. The apparatus according to claim 7, wherein the levers are coupled to respective ones of the links.

9. The apparatus according to claim 7, wherein the levers are coupled to the frame and the anterior floating lens complex such that the levers move the anterior floating lens complex by a first distance with respect to the posterior support structure, when the anterior support structure moves a second distance with respect to the posterior support structure, the first distance greater than the second distance.

10. The apparatus according to claim 7, wherein the links are configured to provide spring functionality that tends to push the anterior support structure away from the posterior support structure.

11. The apparatus according to claim 1, wherein the frame is configured to assume locked and unlocked states.

12. The apparatus according to claim 11, wherein the frame is configured to automatically transition from the locked state to the unlocked state after a period of time after implantation of the AIOL implant in an eye.
